(19) United States
(12) Patent Application Publication
(10) Pub. No.: US 2013/0228997 A1
(43) Pub. Date: Sep. 5, 2013

(54) MOVABLE IV STAND

(71) Applicants: Nobuharu Fukuhara, Osaka-shi (JP); Kazunari Ogawa, Osaka-shi (JP); Shunichi Takamatsu, Osaka-shi (JP)

(72) Inventors: Nobuharu Fukuhara, Osaka-shi (JP); Kazunari Ogawa, Osaka-shi (JP); Shunichi Takamatsu, Osaka-shi (JP)

(73) Assignee: FUJIKOUGYOU CO., LTD., Osaka-shi, Osaka (JP)

(21) Appl. No.: 13/813,193

(22) PCT Filed: Apr. 14, 2011
(86) PCT No.: PCT/JP2011/059773

§ 371 Date: Jan. 30, 2013

(30) Foreign Application Priority Data
Nov. 25, 2010 (JP) 2010-277459

(51) Int. Cl. A61M 5/14

(52) U.S. Cl. CPC A61M 5/1415
USPC 280/304.1

(57) ABSTRACT

Provided is a movable IV stand that allows a patient to walk safely and easily and that also provides a patient in a wheelchair with the possibility of coupling the stand to the wheelchair in a safe, reliable, speedy and flexible manner. For the ease of walking, the base of the movable IV stand has a plan-view shape which is open on the proximal side and a plurality of movable casters are attached to the bottom face thereof. For allowing the coupling to a wheelchair, a joint metal unit is provided to be height-adjustable to a lower portion of a fixed support post or the base of the movable IV stand, thus allowing flexible and movable coupling to the footrest of the wheelchair.

### TECHNICAL FIELD

The present invention relates to an arrangement of a movable IV (intravenous) stand that allows speedy and easy coupling to a wheelchair and that has a leg shape which allows movement thereof simultaneous with a movement of the wheelchair and allows also reliable and safe movement thereof at the time of walking.

### BACKGROUND ART

Many of the conventional movable IV stands are provided with casters for moving that are mounted to a four or five leg base, and a center support post for holding clip infusion solution is provided at the center among the base legs. When a patient receiving infusion walks, the legs or the casters often hinder the walking. Especially, in a gait training given in a post-surgery recovery period, safe walking is not possible; and with occurrence of change in the center of gravity due to attachment of various infusion components thereto, the support post tends to tilt or trip forwardly, thus preventing safe walking with infusion. When an infusion is to be effected for a patient in a wheelchair, a special wheelchair would be needed which has the infusion support post to be set on one lateral side of the wheelchair. However, due to the deviation of the gravity center of the wheelchair to one side, maneuvering was difficult and mounting of many medical/infusion components was infeasible. While a patient in a wheelchair moves, the footrest of the wheelchair is flipped up during the movement, and the patient places his/her feet on the legs of the IV stand for connection to this IV stand and holds the support post of the IV stand with his/her hand, while a nurse or a caregiver maneuvers the wheelchair from behind. For and in riding on an elevator, both the nurse or caregiver and the patient would hold the IV stand against its falling. There is no movable IV stand available that can be coupled in a safe, easy and flexible manner to either the front or the rear of any one of wheelchairs of various sizes and that can be moved in unison with the wheelchair when a patient or a nurse or a caregiver maneuvers the wheelchair with setting the stand to the front or rear thereof, so that the nurse or caregiver can concentrate only on maneuvering of the wheelchair. The IV stand disclosed in Patent Document 2 is burdensome for the patient. The movable IV stand disclosed in Patent Document 4 allows its coupling to the rear of a wheelchair, but does not allow its coupling selectively to either the front or rear of a wheelchair a speedy and easy manner. The IV stand disclosed in Patent Document 3 is not wheelchair-compatible. Further, with the conventional movable IV stands, since a plurality of medical instruments for use in the post-surgery recovery period, infusion components, etc. are attached to a single support post along its vertical direction, the position of the gravity center is high, so that the stand tends to trip over and forward visibility during walk is so poor as to be unable to secure safety in the walk. Furthermore, after the use, due to the five or four-leg shape thereof, the IV stand would occupy a large storage space inside a hospital.

### PRIOR-ART DOCUMENTS

### Patent Documents

Patent Document 1: U.S. Pat. No. 5,704,577

Patent Document 2: Japanese Registered Utility Model No. 3161833

Patent Document 3: Japanese Unexamined Patent Application Publication (Translation of PCT Application) No. 2010-514534

Patent Document 4: Japanese Unexamined Patent Application Publication No. 2009-142407

### SUMMARY OF THE INVENTION

### Object to be Achieved by Invention

In view of the above, the object of the present invention is to provide a movable IV stand that provides a patient in a wheelchair with the possibility of safe/reliable, speedy and flexible coupling between the IV stand and the wheelchair and secures safety and readiness also in walking and that effectively resists tripping-over even when a plurality of medical instruments, infusion components are attached thereto and provides readiness in their attachment and that moreover occupies less storage space in a hospital due to the possibility of nested storage.

### Solution for Achieving the Object

For ensuring sufficient space for walking for a patient receiving infusion, the base of the movable IV stand has a plan-view shape which is open on the proximal side and a plurality of movable casters are attached to the bottom face thereof. A fixed support post is mounted erect on the base, with an approximately upper half portion thereof being disposed on the proximal side and a lower half portion thereof being disposed on the distal side, and a vertically adjustable handle portion is fixedly attached to the upper half portion of the fixed support post, thereby to ensure space for the lower body of the patient and to facilitate his/her walking. And, even when a plurality of infusion components are attached to the upper portion of the fixed support post, as the center of gravity as seen in the lateral view is located at the center of the base, safety is ensured and tripping can be effectively prevented. For a patient in a wheelchair, when the movable IV stand is set to the front of the wheelchair in tandem, for coping with various positions, sizes and angles of the footrest of the wheelchair, a horizontal support pipe having opposed ends thereof bent downwards is fixedly attached to the upper face at the pipe center of left and right opposed sides of the base of the movable IV stand of the present invention and a fixed support post is mounted erect at the center of the horizontal support pipe. A pivot shaft is fixedly attached to the lower portion of the fixed support post, with the pivot shaft extending parallel with the horizontal support pipe and being vertically movable. A joint metal unit is engaged with the pivot shaft. When the IV stand is not to be coupled to a wheelchair, the joint metal unit is pivotally latched to a vertical upper position of the pivot shaft. Whereas, when the IV stand is to be coupled to a wheelchair, the joint metal unit can be pivoted to come into engagement with the footrest of the wheelchair on the proximal side. For coping with the height and angle of the footrest, the position of the pivot shaft of the joint metal unit will be vertically adjusted and fixed. As for the depth-wise dimension of the footrest, a lateral rod member provided at the center between left and right rod members of the joint metal unit provides fixation while allowing mobility in the fore/aft direction. Further, when the IV stand is to be set to the rear of the wheelchair in tandem, the joint metal unit is pivotally latched upwards and the base of the movable IV stand of the invention is inserted into the body frame of rear wheels of the wheelchair and the handle height position of the movable IV stand is adjusted into close vicinity of the handle of the wheelchair. As a result, a nurse or a caregiver can operate both handles of the movable IV stand and of the wheelchair with ease. For readiness of attachment of an infusion component to the stand, from an approximately ring-like metal component that can be fixed to the fixed support post to be vertically adjustable relative thereto, an angular hook-shaped pipe is disposed in the V-form as seen in the plan view and an infusion component holder member is caused to project downwards therefrom. Further, for allowing compact storage in a hospital, the base of the stand has a shape that is open on the proximal side as seen in the plan view, so as to allow nested storage of the movable IV stand.

### Effects of the Invention

The inventive movable IV stand provides a patient in a wheelchair with the possibility of easy coupling thereof to both the front and the rear of the wheelchair. And, in the case of its attachment to the front, flexible engagement and speedy coupling to footrests of various designs of wheelchair are possible, so that the patient can move on his/her own. Also, in case the patient is unable to maneuver the wheelchair by him/herself, a nurse or a caretaker can concentrate on maneuvering of the wheelchair. Further, in case the IV stand is coupled to the rear of a wheelchair in tandem, with appropriate adjustment of the handle position of the movable IV stand, the handle of the stand and the handle of the wheelchair can be brought into close vicinity to each other, so that the nurse or caregiver can operate the handle of the wheelchair and that of the stand simultaneously. For a patient who needs a gait training, lower leg clearance can be secured, so that the patient can walk easily and safely and without risk of tripping over, and the gait training can be carried out simultaneously with infusion operation. With attachment of side frames to the left and right opposed sides of the stand a plurality of medical, infusion components for a patient in a post-surgery recovery period can be attached to the lower positions of the fixed support post. And, as these components can be set to lower positions of the fixed support post, the center of gravity is low such that safety against tripping over can be ensured. Further, as the infusion component holder member comprises a pipe having a V-shape in its plan view and projecting downward, the plurality of infusion components can be attached thereto in juxtaposition, so that the center of gravity can be further lowered for greater safety against tripping-over. Further, due to the arrangement that allows nesting storage of the legs and the support post body, storage space saving effect can be achieved also.

### BRIEF DESCRIPTION OF THE DRAWINGS

FIG. 1 is a perspective view of a movable type IV stand according to Embodiment 1,

FIG. 2 is a plan view of the movable type IV stand according to

Embodiment 1,

FIG. 3 is a front view of the movable type IV stand according to Embodiment 1,

FIG. 4 is a side view of the movable type IV stand according to Embodiment 1,

FIG. 5 is a perspective view showing a base and a fixed support post in Embodiment 1,

FIG. 6 is a perspective view of a movable type IV stand according to Embodiment 2,

FIG. 7 is a plan view showing a base and a compact handle in Embodiment 2,

FIG. 8 is a detailed side view showing a stopper of a joint metal unit pivotal shaft,

FIG. 9 shows a joint metal unit,

FIG. 10 shows a fixed type joint metal unit,

FIG. 11 is a perspective view showing coupling between the movable IV stand of Embodiment 1 and a wheelchair,

FIG. 12 is a perspective view showing in mid-course of coupling between the movable IV stand of Embodiment 2 and a wheelchair,

FIG. 13 is a view showing a rear position coupling of a wheelchair and Embodiment 1,

FIG. 14 shows nesting storage of a plurality of the movable IV stands according to Embodiment 1, and

FIG. 15 shows nesting storage of a plurality of the movable IV stands according to Embodiment 2.

### EMBODIMENTS OF THE INVENTION

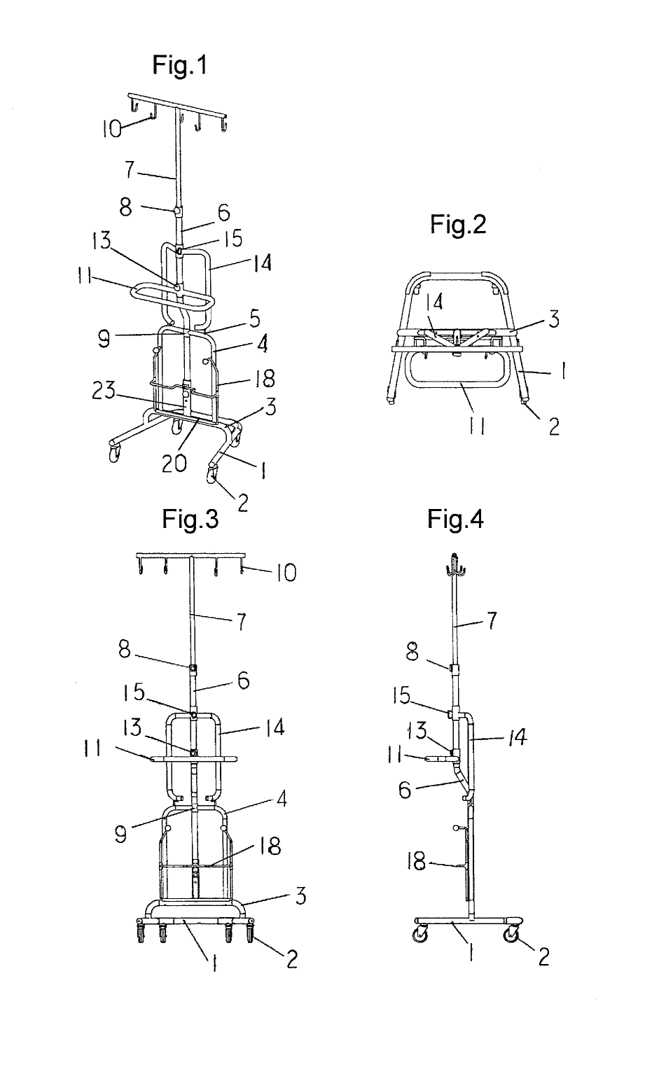

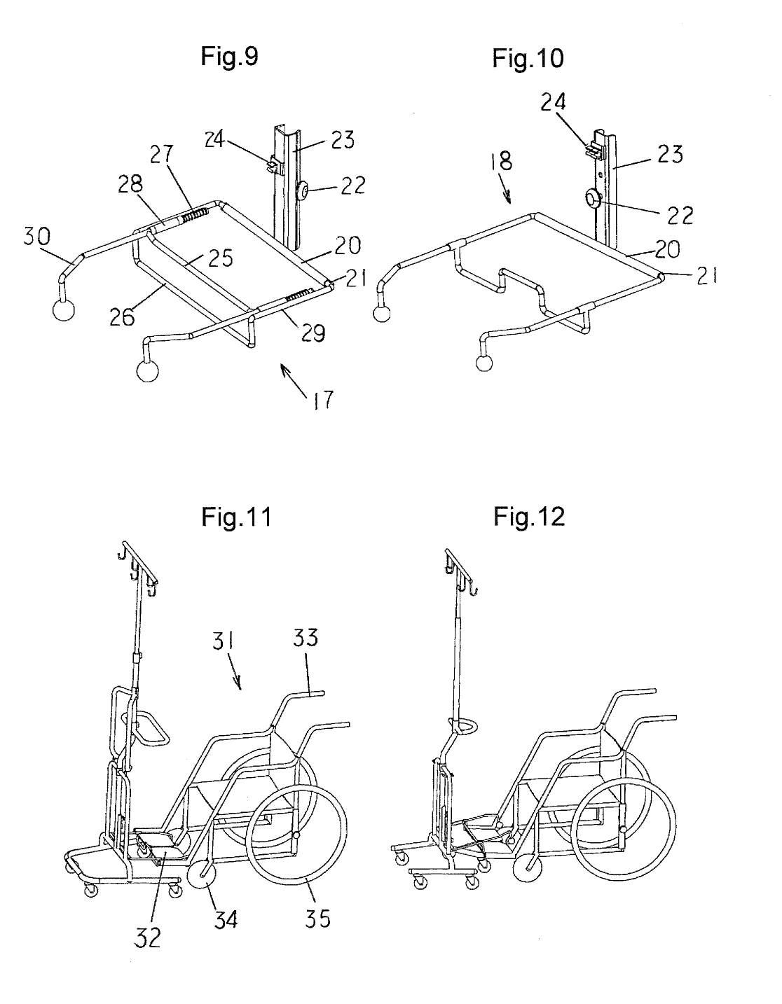

FIGS. 1 through 4 show Embodiment 1 suitable for a movable IV stand according to the present invention. FIG. 1 is a perspective view of the movable IV stand, FIG. 2 is a plan view of the same, FIG. 3 is a front view of the same, and FIG. 4 is a side view of the same, respectively. FIG. 6 shows a movable IV stand according Embodiment 2 of the present invention including a compact type base and a compact handle. FIG. 7 is a plan view showing its base, FIG. 8 is a detailed view of a stopper 21 for a pivot shaft 20 of a joint metal unit 17, 18, FIG. 9 shows the joint metal unit 17 to be coupled to a footrest of a wheelchair, and FIG. 10 shows the fixed type joint metal unit 18.

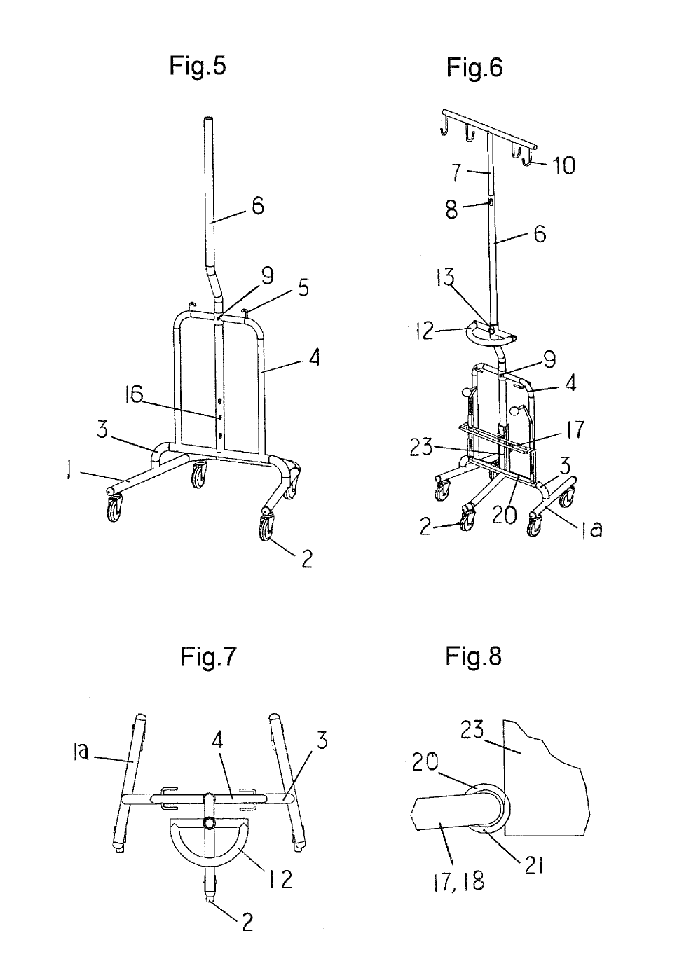

Referring to the basic configuration of the movable IV stand relating to Embodiment 1, as shown in FIG. 1, the basic configuration of the stand includes a base 1, casters 2, a horizontal support pipe 3, a fixed support post 6, a side frame 4, a handle 11, a movable support post 7, an infusion component holder member 14, the joint metal unit 17 or the fixed type joint metal unit 18, a pivot shaft 20 for its attachment, and a pivot shaft fixing channel member 23. The base 1 has a plan view shape that is open on the proximal side and in Embodiment 1, the base 1 is provided as an approximately inverted U-shaped pipe member in the plan view, with its proximal portion being opened in the form of inverted V-shape with an opened apex, and to the bottom of the base 1, there are mounted the four movable casters 2, two of them being provided on the proximal side in left/right symmetry and the other two of them being provided on the leading end or distal side on the opposite lateral sides. In FIG. 5, the horizontal support pipe 3 having opposed ends thereof bent downwards is fixedly attached to the upper faces of the center portions of left and right pipes of the base 1, and at the center of this horizontal support pipe 3, there is mounted erect the fixed support post 6. The fixed support pipe 6 is inserted in a fixed support pipe support ring 9 and guide pipes 28 mounted on the upper faces of the center portions of the horizontal support pipe 3 and fixed thereto with screws, so that the fixed support pipe 6 is installed vertically. Further, the fixed support post 6 is bent by about 6 cm to the proximal side at a portion thereof slightly downward from the center thereof and then bent vertically upward, thus extending parallel with the fixed support post 6 disposed downwards. With the above-described arrangements, the center of gravity as seen in the plan view is located at the approximate center of the four casters 2 mounted to the base 1 and also there is secured a clearance or free space for the lower body of the walker on the proximal side. From the upper portion of the bent lower fixed support post 6, the side frame 4 extends horizontally by about 15 cm along the left and right directions and then bent downwards to be fixedly attached to the opposed ends of the horizontal support pipe 3. As shown in FIG. 5, at the left and right horizontal opposed ends of the side frame 4, side frame hooks 5 are provided for allowing hooking thereto of medical instruments and holding faces therefor are provided in the forward lower face of the fixed support post 6. The side frame hook 5 is configured also for allowing hooking thereon of an elongate tube of the medical instrument in order to prevent accidental entanglement thereof with the casters 2.

FIG. 6 shows a movable IV stand having a base and a compact handle 12. The base of Embodiment 2 is formed by cutting the joint portion at the leading end of the base of Embodiment 1 to form a V shape with an open (separated) apex, and the proximal side portions of the left and right pipes are formed shorter as compared with Embodiment 1 for reduction in the width dimension, and one leg is caused to extend from the proximal side center of the horizontal support pipe 3 toward the proximal side, and the bottom face of the leading end thereof is formed flush with those of the other four legs, and the casters 2 are attached to the bottom faces of the respective leading ends. Further, as shown in FIG. 7, the closing angle between the leading ends of the left and right pipes is set to be about 15 degrees smaller than that of a regular pentagon, thus forming an irregular pentagon. The base having this irregular pentagonal shape provides stability effective against tripping in the fore/aft direction, with securing sufficient left-right clearance for walking on the proximal side for readiness of walking, and provides also improvement in the compactness in the lateral width direction, with the depth dimension being kept substantially intact.

As shown in FIG. 1, FIG. 3, FIG. 4 and FIG. 5, to the proximal side lowermost portion of the fixed support post 6, the pivot shaft 20 is fixed in parallel with the horizontal support pipe 3 to be vertically adjustable, for allowing pivoting of the joint metal members 17, 18 to the proximal side. As the pivot shaft fixing channel member 23 is fixed to a plurality of threaded holes 16 formed at the lower portion of the fixed support post 6 or fastened to the pipe surface of the fixed support post 6 with a threaded handle, adjustment and fixing are done to cope with various heights and angles of the footrest 32 of the wheelchair 31. Referring now to the basic configuration of the joint metal unit 17, as shown in FIG. 9, to the pivot shaft 20 fixedly attached to the lower end of the pivot shaft fixing channel member 23, left and right rod members 29 are engaged and movable left and right rod members 30 are engaged with the guide pipes 28 fixedly attached to the inner sides of the left and right rod member 29 and constantly urged by compression springs 27 toward the pivot shaft 20. When the IV stand is not to be coupled to a wheelchair, the joint metal unit 17 is flipped up to be positioned perpendicularly upward, and with pivotal latched connection between a joint metal unit catch 24 and a lateral rod member 25, there can be provided the lower body clearance for not interfering with walking. Whereas, when the IV stand is to be coupled to a wheelchair, after release of the pivotal latch of the joint metal unit 17, the coupling is established with pivotal engagement between the footrest 32 of the wheelchair 31 on the proximal side and the vertical rods on the opposed sides of the fixed horizontal rod member 26 and the L-shaped curved portions of the movable left and right rod members 30. As shown in FIG. 8, the pivot shaft 20 is provided, at its opposed ends, with the pivot stoppers 21 for preventing pivotal fall of the joint metal unit 17, 18 to an angle smaller than or equal to the minimal angle.

As shown in FIG. 9, in the joint metal unit 17, the left and right rod members 29 engaged with the pivot shaft 20 and the fixed lateral rod member 26 are formed of a single rod member and the guide pipes 28 are fixedly attached to the inner sides of the left and right rod members 29 respectively, and the movable left and right rod members 30 extend along the guide pipes 28 and the compression springs 27 are inserted thereinto and the leading ends thereof on the side of the pivot shaft are fixed with screws, while the leading ends on the opposite sides are bent downward in the L-shape and spherical balls are attached to the terminal ends thereof. In order to comply with the depth dimension of the footrest 32 of the wheelchair 31, the movable-side lateral rod member 25 can be adjusted in the fore/aft direction and compressed by the compression spring. FIG. 10 shows a further embodiment relating to a fixed type joint metal unit comprised of at least three rod members consisting of left and right rod members and a lateral rod member. Each of the left and right rod member has its leading end bent downwards in the L-shape, with a spherical ball being attached thereto, for facilitating engagement with the footrest 32 of the wheelchair 31 and ensuring safety for the foot and leg of the patient at the time of the engagement. As shown in FIG. 9 and FIG. 10, the left and right L-shaped portions are bent inwards toward each other by about 30 degrees as seen in the plan view. This arrangement is provided for avoiding interference between the leading end of the outer wheel and the L-shaped portion of the joint metal unit 17 or the fixed type joint metal unit 18 when front wheels 34 of the wheelchair 31 are pivoted forwardly in association with a fore/aft movement of the wheelchair 31. The above-described arrangements serve to allow flexible coupling in accordance with any possible difference in the size, angle, setting height of the footrest 32 of the wheelchair 31. Selection between the adjustable type shown in FIG. 9 and the fixed type shown in FIG. 10 can be made by the user at the time of installment. As the inner sides of the left and right L-shaped leading ends of the joint metal unit 17 and the fixed type joint metal unit 18 are separated from each other rather than being joined to each other, at the time of coupling, the patient in a wheelchair can carry out the coupling or decoupling operation without having to lift his/her legs from the footrest 32. As the joint metal unit 17 and the fixed type joint metal unit 18 are vertically movable relative to the footrest 32 of the wheelchair, the coupling adaptability between the movable IV stand of the present invention and the wheelchair is not rigid engagement, but flexible engagement, so that the stand can follow movement of the wheelchair in a flexible manner, and the stand does not provide or receive effect from unevenness on the floor surface. Further, when the wheelchair moves forwardly and then makes a sudden stop, the IV stand of the invention will be subject to an inertial force at its front portion. However, lower pivotal movement is stopped by the pivot stopper 21 of the joint metal unit 17 or the fixed type joint metal unit 18. Hence, no reduction occurs in the forward tripping angle relative to the fixed support post 6 of the inventive IV stand, so there occurs no problem of forward tripping.

As shown in FIG. 1, FIG. 3 and FIG. 4, in order to comply with the body condition of the patient, to the upper half portion of the fixed support post 6, there are provided grip portions formed of ring-shaped pipes each having a total width of 38 cm on the left and right opposed sides of the handle adjustment pipe for allowing vertically adjustable fixation and these are engaged with the fixed support post 6 and fixed in position with a handle vertical adjustment knob 13.

As shown in FIG. 1, within the hollow column of the upper half portion of the fixed support post 6, the movable support post 7 is disposed to be vertically adjustable relative thereto; and at the upper end of this movable support post 7, there is provided a T-shaped horizontal bar having hooks 10 projecting downwards for allowing hooking of an infusion solution bag. The fixed support post is provided, at its upper end, with a threaded portion for allowing the height adjustment of the movable support post 7 and the engagement and fixation between the fixed support post 7 and the movable support post 6 and these are fixed to each other with a movable support post fixing knob 8.

As shown in FIG. 1, FIG. 3 and FIG. 4, the infusion component holder member 14 that can be fixed to the fixed support post 6 to be vertically adjustable relative thereto is comprised of an elongate pipe member extending from the left and right sides of the approximately ring-shaped metal member in the form of V-shape in the plan view and projects downwards. And, this holder member 14 is engaged with the fixed support post 6 and fixed thereto with a holder member vertical adjustment knob 15. When a plurality of infusion components are attached to two left and right pipes rather than to a single pipe as is the case with the conventional IV stand, the position of center of gravity thereof can be lower and it becomes also possible to provide better visibility for the walker or the driver of the wheelchair for ensuring safe movement thereof. The V-shaped arrangement allows disposing of the handle 11 and the compact handle 12 without interference in their height positions with the infusion components.

In case the inventive movable IV stand is to be coupled to the front of the wheelchair 31, as shown in FIG. 11 and FIG. 12, the joint metal unit 17 or the fixed type joint metal unit 18 will be released from the upper joint metal unit catch 24 and then pivotally tilted forwards to come into engagement from the above with the footrest 32 of the wheelchair 31 for coupling therewith. In this, the positional relationship between the wheelchair and the movable IV stand of the invention will be such that the proximal pipe ends of the base 1 and the front wheel casters 34 of the wheelchair 31 will not come into contact with each other even when being pivoted. Further, due to the flexible movable coupling to follow the movement of the wheelchair 31, the IV stand will not be inadvertently decoupled from the footrest 32 unless the joint metal unit 17 or the fixed type joint metal unit 18 is intentionally lifted up from the footrest 32 and released.

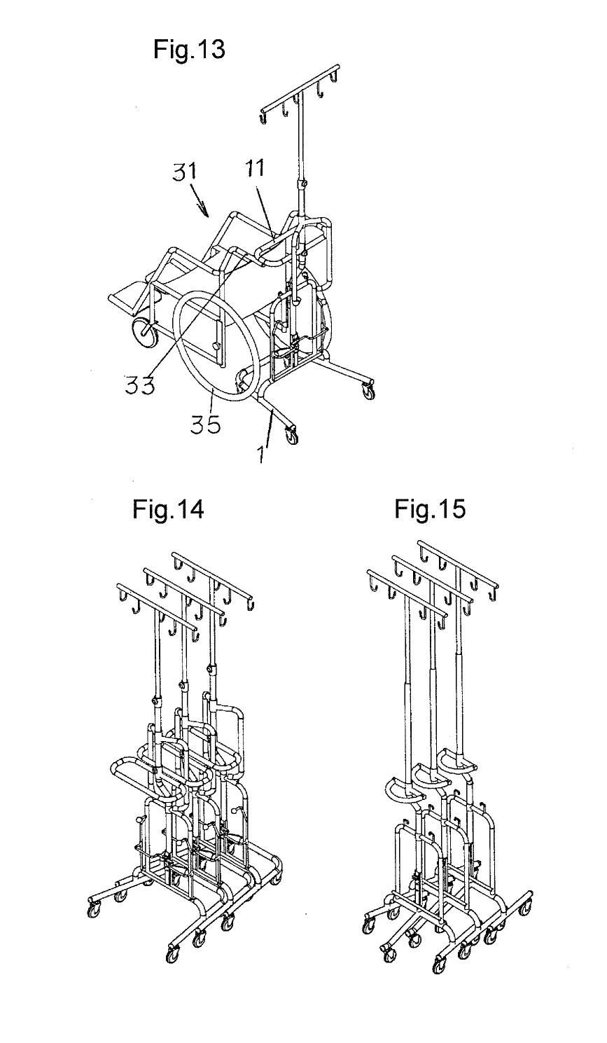

In case the body of the inventive stand is to be coupled to the rear of the wheelchair 31, as shown in FIG. 13, the base 1 of the inventive stand will be inserted into the main body frame of the rear side of the wheelchair 31 or between a pair of large wheels 35 of the wheelchair 31 and the handle 11 will be brought closer in accordance with the height of the handle 33 of the wheelchair 31. With some types of wheelchairs, depending on the positional relationship and/or size thereof, the handle 11 can be adjusted in its position to an approximately 180 degrees pivoted position as shown in FIG. 13, so as to bring the handles of the stand and the wheelchair closer to each other for allowing simultaneous maneuvering thereof.

For a patient who can walk, the inventive IV stand can be used, with detaching the entire joint metal unit from the fixed support post 6. Further, for a patient who does not need the infusion component holder 14, this holder 14 can be detached for achieving weight reduction and greater facility in movement.

The configuration according to the present invention allows nesting storage, so that for the storage after use in a hospital or the like, a plurality of the stands can be stored in neat tandem as shown in FIG. 14 and FIG. 15, with upward flipping of the joint metal unit 17 or the fixed metal unit 18 and pivotal latched engagement thereof with the joint metal unit catch 24.

### INDUSTRIAL APPLICABILITY

While the present invention provides one proposal for a method of coupling between a medical movable IV stand and a wheelchair of various sizes, but can be used equally, in an inexpensive, simple and flexible manner for various instruments which can be coupled to a wheelchair smoothly and easily so that a busy nurse or a caregiver in a hospital can concentrate on the main task.

### DESCRIPTION OF REFERENCE NUMERALS/MARKS

1 base

1a base of Embodiment 2

2 caster

3 horizontal support pipe

4 side frame

5 side frame hook

6 fixed support post

7 movable support post

8 movable support post fixing knob

9 movable support post support ring

10 suspension hook

11 handle

12 compact handle

13 handle vertical adjustment knob

14 infusion component holder member

15 infusion component holder member vertical adjustment knob

16 joint metal unit height adjustment hole

17 joint metal unit

18 fixed type joint metal unit

20 pivot shaft

21 pivot shaft stopper

22 joint metal unit vertical adjustment knob

23 pivot shaft fixing channel member

24 joint metal member catch

25 lateral rod member

26 fixed lateral rod member

27 compression spring

28 guide pipe

29 left/right rod member

30 movable left/right rod member

31 wheelchair

32 footrest

33 wheelchair handle

34 wheelchair front wheel caster

35 large wheel

1. An IV stand movable on a floor surface, comprising:
a plurality of movable casters attached to a bottom face of a base;
a fixed support post mounted erect on a center of the base;
a vertically movable pivot shaft attached to a lower portion of the fixed support post or the base; and
a joint metal unit engaged with the pivot shaft for allowing pivotal engagement with a footrest of a wheelchair on a proximal side thereof.

2. The movable IV stand according to claim 1, wherein the joint metal unit is comprised of at least three rod members including left and right rod members and a lateral rod member;
a leading end of each of the left and right rod members is bent downwards downwardly in an L-shape, so that the lateral rod member interconnecting the left and right rod members can be fixed with possibility of movement in a fore/aft direction.

3. The movable IV stand according to claim 1, wherein the base comprises left and right pipes joined to each other with an opening therebetween on the proximal side in the plan view, via a horizontal support pipe.

4. The movable IV stand according to claim 1, wherein an upper portion of the fixed support post is located on the proximal side and a lower portion thereof is located on a distal side thereof.

5. The movable IV stand according to claim 1, wherein a handle portion provided in the fixed support post is vertically adjustable.

6. The movable IV stand according to claim 4, wherein within a hollow column of the upper portion of the fixed support post, a movable support post is fixedly disposed with a possibility of vertical adjustment.

7. The movable IV stand according to claim 1, wherein, an injection component holder member can be attached to the fixed support post, the injection component holder member comprising an angular hook shaped pipe projecting downwardly on left and right opposed sides from an approximately ring-shaped metal member that can be fixed to the fixed support post to be vertically adjustable relative thereto.
